(19) United States
(12) Patent Application Publication
(10) Pub. No.: US 2016/0279158 A1
(43) Pub. Date: Sep. 29, 2016

(54) USE OF GLUCOSAMINE-ENRICHED PLANT COMPOSITIONS

(71) Applicant: NESTEC S.A., Vevey (CH)

(72) Inventors: Yann Mahe, Sainte Genevieve Des Bois (FR); Pierre Edmond Broun, Tours (FR); Jwanro Husson, Notre Dame d'Oe (FR); Didier Courtois, St-Avertin (FR)

(21) Appl. No.: 15/033,753

(22) PCT Filed: Nov. 4, 2014
(86) PCT No.: PCT/EP2014/073676

§ 371 Date: May 2, 2016

(30) Foreign Application Priority Data
Nov. 5, 2013 (EP) 13191664.5

(51) Int. Cl. A61K 31/7008; A61K 36/28

(52) U.S. Cl. CPC A61K 31/7008; A61K 36/28

(57) ABSTRACT

The invention relates to glucosamine-enriched plant compositions and their use as a biocide, for instance, in the treatment of skin diseases and/or mucosal diseases. The invention also concerns the use of glucosamine-enriched plant compositions for preventing and/or reducing body odors.

### TECHNICAL FIELD

The invention relates to glucosamine-enriched plant compositions and their use as a biocide.

The invention also relates to the use of glucosamine-enriched plant compositions for preventing and/or reducing body odors.

### BACKGROUND OF THE INVENTION

The use of pure glucosamine in the treatment of joint diseases is widely described in the patent as well as in the scientific literature, usually in combination with other compounds or extracts from various natural sources. Pure glucosamine is added as glucosamine hydrochloride or glucosamine sulfate, and comes from shellfish hydrolysis. For example, WO2000/0074696 describes “herbal compositions comprising glucosamine and Trypterygium wilfordii, Ligustrum lucidum and/or Erycibe schmidtii, for treating inflammation or degeneration of joint tissues, e.g. arthritis” where pure glucosamine is mixed with plant preparation, Other patents relate to compositions of plant carbohydrates as dietary supplements where glucosamine is originated from chitin, i.e. once again from shellfish hydrolysis (see for example EP 1 172 041 or EP 0 923 382).

Interestingly, glucosamine is also related to the aging process of skin, which has been characterized mainly by the continuous loss of elasticity and the loss of moisture. Skin aging is reflected by major structural changes and variations in composition. Most notably aged skins have less collagen and glycosaminoglycans compared with young skins. Glycosaminoglycan molecules produced by the skin include hyaluronic acid (poly d-glucuronic acid-n-acetyl-d-glucosamine), chondroitin sulfate, and dermatan sulfate. Hyaluronic acid is produced in higher quantities by the skin cells in response to exfoliation. Hyaluronic add has a large capacity for hydration.

Glucosamine has been shown to significantly improve dryness of the skin and exfoliation. Glucosamine increases the moisture content and improves the smoothness of the skin, These findings suggest that long-term intake of glucosamine is effective in improving moisture content and smoothness of the skin.

It has been shown that oral supplement containing glucosamine lead to a reduction in the number of visible wrinkles and in the number of fine lines in a group of women who took the supplement. The use of an oral supplement containing glucosamine, minerals, and various antioxidant compounds can potentially improve the appearance of visible wrinkles and fine lines.

U.S. Pat. No. 6,413,525 describes methods of substantially exfoliating the skin. In particular, it relates to topically applied compositions containing an amino sugar in the form of N-acetyl-D-glucosamine. N-acetyl-D-glucosamine is known to be a rate-limiting factor in the hyaluronic acid production by living cells. The topical application of glucosamine assists in the continued production of hyaluronic acid.

U.S. Pat. No. 6,992,073 concerns the use of N-acetyl-D-glucosamine as a therapeutic agent for resisting the field planting activity of microorganism and thus prevent from infection.

EP 1 669 077 describes the combination of N-acetyl-D-glucosamine and antibiotics for the preparation of antibacterial drugs.

Glucosamine, 2-amino-2-deoxy-D-glucose, is a naturally occurring derivative of fructose and is an essential component of glycoproteins and proteoglycans, important constituents of many eukaryotic proteins. This is an essential component of mucopolysaccharides and chitin. Glycosaminoglycans (mucopolysaccharides) are large complexes incorporated into connective tissue, skin, tendons, ligaments and cartilage.

Industrial glucosamine is a pure compound obtained from the acidic hydrolysis of chitin from shellfish, a complex carbohydrate derived from N-acetyl-D-glucosamine. As an example, U.S. Pat. No. 6,486,307 describes an improved method for chitin acidic hydrolysis: a method of producing glucosamine hydrochloride from chitin by grinding the chitin to a very fine size and digesting it with concentrated hydrochloric acid. Glucosamine can also be produced from enzymatic hydrolysis of shellfish. As an example, U.S. Pat. No. 5,998,173 describes a process for directly producing N-acetyl-D-glucosamine from chitin utilizing an ensemble of the chitinase family of enzymes to hydrolyze chitin of crustacean shells.

Patents have also been filed protecting microbial fermentation processes where cultivated microorganisms biosynthesize glucosamine. As an example, U.S. Pat. No. 6,372,457 describes a method and material for producing glucosamine by fermentation using a genetically modified microorganism.

All these processes concern the production of pure, extracted glucosamine, in comparison to shellfish extracts.

WO2005/053710 shows that glucosamine can be formed from several raw plant materials by following a special drying process, therefore obtaining glucosamine contents of between 150 mg and 1000 mg per kg of dry matter.

WO2006/120009 describes a process for generating glucosamine from plants wherein fresh plant materials, or re-hydrated dried plant materials or plant extracts, are heated at a temperature comprised between 70° C. and 110° C. for more than 10 h, wherein a glucosamine precursor, such as ammonium sulfate salt, is added to said plant materials, rehydrated plant materials or plant extracts.

WO2006/120007 relates to a process for generating glucosamine from plants similar to the one described in WO2006/120009, wherein a fertilizer acting as glucosamine precursor is added during the cultivation of the plants, before the harvest.

In the context of allergies, food intolerance or other specific expectations (e.g. religion), glucosamine that is formed from plant materials may be required. This type of glucosamine is also environmentally safe, biodegradable and renewable.

Furthermore, there is a need for therapeutic or cosmetic composition for the skin which will not trigger such allergies, food intolerance etc. In particular, there is a need for such compositions in therapeutic field which provide a biocide effect.

The biocide effect may stimulate the innate immune system so as to help prevent or fight against invasion of exogenous pathogens (for instance bacteria or fungi). The epithelial cells are, in particular, defended by antimicrobial peptides or proteins that are part of the innate immune system and are one of the primary mechanisms used by the skin in the early stages of immune defense.

Thus, under normal conditions, antimicrobial peptides or proteins are synthetized, when needed, by the epithelial cells.

However, there is a need to stimulate immune response and, in particular, the synthesis of antimicrobial peptides or proteins to improve or strengthen the barrier function of the skin when deficiencies occur. For example atopic dry skin or wounded skin has an impaired barrier function and thus the innate immune response may not be sufficient to limit bacterial proliferation and infections.

It is therefore an object of the invention to provide a glucosamine-enriched plant composition for use as a biocide for skin and/or mucosal membranes.

### SUMMARY OF THE INVENTION

In a first aspect of the invention there is provided a glucosamine-enriched plant composition for use as a biocide.

In a preferred embodiment said composition is used as a biocide in the treatment of skin diseases and/or mucosal diseases.

In particular, the glucosamine-enriched plant composition for use according to the invention stimulates the synthesis of antimicrobial peptides or proteins of the skin and/or mucosal membranes.

In a preferred embodiment of the invention, the glucosamine-enriched plant composition is a hydroalcoholic plant extract or a hydroalcoholic plant extract of roots.

In another aspect of the invention the glucosamine-enriched plant composition is used for preventing and/or reducing body odors.

### DETAILED DESCRIPTION

The invention provides a glucosamine-enriched plant composition for use as a biocide.

In the present specification, glucosamine, sulfate and ammonium contents are expressed either in percentages based on dry matter or in g per kg of dry matter unless indicated otherwise.

In the present specification, “high amount of glucosamine” or “high glucosamine content” as used therein means that the amount of glucosamine is higher than traces of glucosamine, higher than the amounts in the corresponding fresh (i.e. non-dried) material and higher than any content cited in literature or patents. In particular, glucosamine is present in amounts higher than 0.5% based on dry matter, preferably greater than 5% and, in particular, greater than 10% based on dry matter.

As used herein, the “glucosamine-enriched plant composition” refers to the product obtained through a process for generating glucosamine in a plant material with or without extraction or purification steps. This glucosamine-enriched plant composition comprises a high amount of glucosamine as defined above.

The words “plant” and “plant material”, as used herein, are considered as synonyms. By “plant” or “plant material”, it has to be understood any plant material capable of generating glucosamine according to processes described in this specification. The plant material used can be any part of the plant, e.g. leaves, tubers, fruits, seeds, roots, grains or cell cultures. Said plant material can also be any type of plant extract obtained by any extraction procedure known to the skilled person in the technical field of plant extraction, where said plant extract is capable of generating glucosamine according to the process of the invention.

In this specification, the word “biocide” refers to a compound or a composition which can be used to control any harmful microorganism and/or prevent microbial growth by chemical or biological means. For instance, a biocide activity can be observed for an antimicrobial or an antibacterial agent.

In the present specification, the terms “antimicrobial peptides or proteins” refer respectively to peptides or proteins expressed, in particular, by the keratinocytes when they come into contact with microbial pathogenic agents.

As used in this specification, the words “comprises”, “comprising”, and similar words, are not to be interpreted in an exclusive or exhaustive sense. In other words, they are intended to mean including, but not limited to.

Any reference to prior art documents in this specification is not to be considered an admission that such prior art is widely known or forms part of the common general knowledge in the field.

In a first aspect of the invention there is provided a glucosamine-enriched plant composition for use as a biocide.

The glucosamine-enriched plant composition for use according to the invention may be applied, for example, in the treatment or prevention of skin diseases and/or mucosal diseases.

Skin and/or mucosal diseases caused, for instance, by viruses, bacteria or fungi may disturb the permeability barrier function and can result from dermatological disorders. Said dermatological disorders may be, for example, acne, seborrheic keratosis, melanoma, infectious complications during wound healing, atopic dermatitis, rosacea, eczema, psoriasis, candidiasis, vaginoses, scalp ringworm, skin disorders linked to therapies with antibiotics or antimycotic agents or brought about by hormone disturbances.

The glucosamine-enriched plant composition for use according to the invention may also apply in the treatment and/or prevention of acne and/or dandruff of the scalp.

The glucosamine-enriched plant composition for use according to the invention may also be applied for stimulating the synthesis of antimicrobial peptides or proteins of the skin and/or mucosal membranes.

The glucosamine-enriched plant composition according to the invention may be obtained through any processes for generating glucosamine from plants. In particular, it can be obtained by the process comprising the following successive steps:
placing a plant material and at least one glucosamine precursor, into a receptacle,
closing said receptacle,
heating said receptacle at a temperature of 70° C. to 110° C. for more than 10 h.

Alternatively the heating step, which can be described as a drying step, can consist in a liquid maceration, carried out at the same temperature and time conditions, and then followed by a drying phase.

Glucosamine may be formed, in particular, by the condensation of a nitrogen-containing moiety and a sugar moiety. The glucosamine precursors used as indicated above may be compounds providing the nitrogen-containing moiety needed for the formation of glucosamine. Preferably, they consist in ammonium salts. Examples of such ammonium salts are ammonium nitrate, ammonium sulfate, ammonium acetate, ammonium dihydrogenophosphate or glutamine, among others. The preferred precursor of glucosamine is ammonium sulfate which has shown surprisingly good results in the process according to the invention and in WO2006/120009 and WO2006/120007.

According to the invention, the glucosamine-enriched plant compositions comprise a high glucosamine content and, for example, a glucosamine content higher than 0.5% based on dry matter, preferably greater than 5% and, in particular, greater than 10% based on dry matter.

In a preferred embodiment, the plant material is the roots of the plant.

The plant material may, in particular, be selected from the group consisting of plant species containing sucrose, fructose or inulin such as Cichorium, Daucus, Helianthus, Beta.

In one embodiment, the plant material may be, for example, from root of Chicory (Cichorium intybus), root of carrot (Daucus carota), tuber of Jerusalem artichoke (Helianthus tuberosum), root of beet (Beta vulgaris).

In a preferred embodiment, the plant species is Cichorium intybus used for the production of roots containing inulin, or for the production of Belgian endive in fields or in hydroponic culture systems: i.e. chicon, witloof chicory, witloof, French endive, white endive, Dutch chicory, succory, common chicory, or Italian dandelion is Cichorium intybus. Witloof chicory is the common name used by most horticulturists for the cultivated plant, while Belgian endive is more used for the product ultimately sold in grocery stores to consumers. Endive is the second growth of the bud from the top of a chicory root. The first growth takes place in the field on a plant grown from seed. The second growth takes place out of the field, usually in a building, in the dark. Each root has one main bud leading to the development of the chicon (chicory, endive).

Alternatively, the process for generating glucosamine from plants as described above further comprises the following steps:
opening said receptacle, and
drying the obtained glucosamine-enriched plant composition.

This drying step can be performed through any suitable known methods to the person skilled in the art wherein the temperature remains under about 110° C. in order to avoid degradation of glucosamine. For example, usual dryers can be used. It is also possible to change the pressure conditions.

Preferably, the glucosamine-enriched plant composition according to the invention may be obtained through a process for generating glucosamine from plants as described above which further comprises a step of extracting the obtained glucosamine-enriched plant compositions. This extraction step can consist, for example, in adding said obtained glucosamine-enriched plant compositions in water for about 30 min at room temperature. Alternatively, said obtained glucosamine-enriched plant compositions may be extracted with water:ethanol (50:50) for 60 min at room temperature.

Then the resulting mixture is centrifuged and the supernatant is freeze-dried. Alternatively, said resulting mixture is filtered and the filtrate is further dried.

Preferably, said obtained glucosamine-enriched plant compositions can be extracted with a buffer solution of high pH, for example with a pH equal to or greater than 10 such as a carbonate buffer of pH 10.8.

In such pH conditions, ammonium is transformed into ammonia which is volatile and thus evaporated. Accordingly, the glucosamine-enriched plant compositions so obtained comprise a reduced amount of ammonium residue and, in particular, less than 0.5% of ammonium ions, and preferably less than 0.1%.

Processes for generating glucosamine from plants comprising the step of extracting the obtained glucosamine-enriched plant compositions with a buffer solution of pH equal to or greater than 10 as described above, may further comprise the following steps:
adjusting the pH of the solution to 6.5 to 7,
adding a solution of calcium chloride,
adding ethanol.

For example, concentrated hydrochloric acid can be used in order to adjust the pH of said solution to 6.5 to 7.

The addition of calcium chloride leads to the formation of calcium sulfate complex which precipitates by adding ethanol.

The glucosamine-enriched plant compositions thus obtained after extraction comprise reduced amounts of residual ammonium ions and sulfate ions.

High amounts of ammonium ions and/or sulfate ions in the obtained glucosamine-enriched plant materials or plant extracts may be detrimental depending on the application's field. Indeed, high amounts of ammonium and sulfate ions are known as being aggressive for the skin, eyes and mucous membranes and should thus be avoided, in particular, in cosmetic or therapeutic field, for example, for the preparation of topical creams.

In another aspect of the invention, the glucosamine-enriched plant composition may comprise at least 0.5% of glucosamine based on dry matter and preferably greater than 5% of glucosamine and, in particular, greater than 10% of glucosamine based on dry matter.

In particular, said glucosamine-enriched plant compositions comprise less than 0.5% of ammonium ions based on dry matter. Preferably, it contains less than 0.5% of ammonium ions and less than 0.5% of sulfate ions based on dry matter, in particular, less than 0.1% of ammonium ions and less than 0.2% of sulfate ions based on dry matter.

In a further embodiment, the glucosamine-enriched plant composition for use according to the invention is such that said glucosamine-enriched plant composition is in the form of granules, powders, tablets, coated tablets, capsules, suppositories, syrups, emulsions, gels, ointments, suspensions, creams, aerosols, droplets or injectable forms.

In another embodiment, the glucosamine-enriched plant composition for use according to the invention is for topical use. It thus may provide a local effect.

The glucosamine-enriched plant composition for use according to the invention may also be for oral administration. In such a case, it may provide a systemic action.

In a further embodiment, the glucosamine-enriched plant composition according to the invention may also comprise nonpathogenic microorganisms such as, for example, Vitreoscilla filiformis. This latter bacterium has the ability to balance cutaneous immune-homeostasis.

In another aspect of the invention, there is provided the use of a glucosamine-enriched plant composition for preventing and/or reducing body odors. These unpleasant odors are, for example, associated with the proliferation of skin microorganisms in the confined areas of the body, in particular the axillary zone or the feet. For this type of application, the glucosamine-enriched plant composition according to the invention may then be formulated in deodorant compositions.

The invention is further described with reference to the following examples. It will be appreciated that the invention as claimed is not intended to be limited in any way by these examples.

The skilled person will recognize many variations in these examples to cover a wide range of formulas, ingredients, processing, and mixtures to rationally adjust the naturally occurring levels of the compounds of the invention for a variety of applications.

### EXAMPLE 1

### Preparation of a Glucosamine-Enriched Plant Composition (Aqueous Plant Extract)

200 g of commercial dried dices of chicory roots (0.5×0.5×0.5 cm) are soaked in a solution of ammonium sulfate 0.5 M for 3 h. After draining, dices are placed in a container with a lid just placed above. This closed container is then placed in an oven (Binder) at a temperature of 75° C. for 48 h.

Then the container is open and the dices are spread out (10 cm dice layer) for drying in the same oven, keeping the temperature at 75° C.

2 g of powdered processed chicory roots are extracted with 200 ml of water for 30 min at room temperature. After centrifugation, the supernatant is freeze-dried.

The resulting glucosamine-enriched plant composition is thus an aqueous plant extract (hereafter referred to as Compound A).

### EXAMPLE 2

### Preparation of a Glucosamine-Enriched Plant Composition (Hydroalcoholic Plant Extract)

200 g of commercial dried dices of chicory roots (0.5×0.5×0.5cm) are soaked in a solution of ammonium sulfate 0.5 M for 3 h. After draining, dices are placed in a container with a lid just placed above. This closed container is then placed in an oven (Binder) at a temperature of 75° C. for 48 h.

Then the container is open and the dices are spread out (10 cm dice layer) for drying in the same oven, keeping the temperature at 75° C.

20 g of powdered processed chicory roots are extracted with 100 ml of water:ethanol (50:50) for 60 min at room temperature. After centrifugation, the ethanol is removed from the liquid phase using a rotary evaporator and the aqueous solution is freeze-dried.

The resulting glucosamine-enriched plant composition is thus a hydroalcoholic plant extract (hereafter referred to as Compound B).

### EXAMPLE 3

### Measurement of the Biocide Activity on Aqueous and/or Hydroalcoholic Plant Extract

Both dried plant extracts obtained at examples 1 and 2 (i.e. respectively aqueous and hydroalcoholic plant extracts) are used to prepare the following test compounds:
Compound A: the resulting glucosamine-enriched plant composition of example 1 which is an aqueous plant extract, in both concentrations 5 μg/ml and 50 μg/ml;
Compound B: the resulting glucosamine-enriched plant composition of example 2 which is a hydroalcoholic plant extract, in both concentrations 5 μg/ml and 50 μg/ml.

Each test compound is applied for 24 hours, at both indicated concentrations (i.e. 5 μg/ml or 50 μg/ml), alone or in combination, to normal human epidermal keratinocytes (NHEK) in culture in vitro at 37° C. and 5% CO₂.

The culture supernatants are subsequently removed and the carpets of cells are rinsed with a solution of phosphate buffered saline (PBS) and then placed in sterile RNAse-free tubes and immediately frozen at −80° C.

The expression of the precursors of synthesis of the proteins of interest (i.e. S100A7) was evaluated by RT-Q-PCR (Reverse Transcription Quantitative Polymerase Chain Reaction) using the messenger RNAs extracted from the cells for each treatment conditions.

Results are expressed by comparison with the corresponding untreated control conditions.

### Reverse Transcription:

extracting the total RNA of each sample using TriPure Isolation Reagent® according to the protocol recommended by the supplier;
eliminating the traces of potentially contaminating DNA by treatment with the DNA-free system (Ambion);
carrying out the mRNA reverse-transcription reaction in the presence of the oligo(dT) and of the Superscript II enzyme (Gibco).

### Quantitative PCR:

The PCR reactions were performed by quantitative PCR with the “Light Cycler” system (Roche Molecular Systems Inc.) and according to the procedures recommended by the manufacturer. The biomarker used in this study is S100 calcium binding protein A7 (i.e. S100A7) which is considered as a representative antimicrobial protein (AMP), the impaired expression of which in the skin contributes to increased susceptibility to skin infections (Gläser et al., The Antimicrobial Protein Psoriasin (S100A7) Is Upregulated in Atopic Dermatitis and after Experimental Skin Barrier Disruption, J. Invest Dermatol. (2009) 129, 641-649). The mean value of the relative expression is expressed in Arbitrary Units calculated from the values of two independent PCR cycles according to the following formula:

(l/2^{number of cycles})×10⁶.

The results for the differential expression of the biomarker S100A7 relative to untreated control conditions are as follows:

| | Compound | | | | Compound A + | Compound A + |
|---|---|---|---|---|---|---|
| | A | | Compound B | | Compound B | Compound B |
| Official | 5 | 50 | 5 | 50 | (5 μg/ | (50 μg/ |
| Symbol | μg/ml | μg/ml | μg/ml | μg/ml | ml each) | ml each) |
| S100A7 | ×1.6 | ×1.3 | ×1.5 | ×3.9 | ×1.3 | ×6.0 |

Therefore, as indicated in the Table above, in response to glucosamine-enriched plant composition of compound A and B alone or in combination at two different concentrations (i.e. 5 μg/ml and 50 μg/ml), the induction of mRNA encoding S100A7 whose presence is linked with a biocide effect, is clearly observed in all conditions in NHEK.

It is also noted that, at 50 μg/ml, the glucosamine-enriched plant composition of compound B, which is a hydroalcoholic extract, provides better results compared to the aqueous extract (i.e. compound A).

1. A biocide comprising glucosamine-enriched plant composition.

2. A method for the treatment or prevention of skin diseases and/or mucosal diseases comprising administering a composition comprising a glucosamine-enriched plant composition to an individual in need of same.

3. Method according to claim 2 for stimulating the synthesis of antimicrobial peptides or proteins of the skin and/or mucosal membranes.

4. Method according to claim 2 for the treatment and/or prevention of acne and/or dandruff of the scalp.

5. Method according to claim 2 wherein the glucosamine-enriched plant composition comprises at least 5 g of glucosamine per kg of dry matter.

6. Method according to claim 2 wherein the plant is a part of plant selected from the group consisting of leaves, tubers, fruits, seeds, roots, grains and cell cultures.

7. Method according to claim 2 wherein the glucosamine-enriched plant composition is a hydroalcoholic plant extract.

8. Method according to claim 2, wherein the glucosamine-enriched plant composition is a hydroalcoholic plant extract of roots.

9. Method according to claim 2 wherein the glucosamine-enriched plant composition is in a form selected from the group consisting of granules, powders, tablets, coated tablets, capsules, suppositories, syrups, emulsions, gels, ointments, suspensions, creams, aerosols, droplets and injectable forms.

10. Method according to claim 1 wherein the composition is applied topically.

11. Method according to claim 2 wherein the composition is ingested orally.

12. Method according to claim 9, wherein the glucosamine-enriched plant composition further comprises nonpathogenic microorganisms.

13. Method according to claim 2 wherein the plant is Cichorium intybus or Chicory.

14. Method for preventing and/or reducing body odors comprising using a composition comprising glucosamine-enriched plant.

15. Method according to claim 14 wherein the glucosamine-enriched plant composition comprises at least 5 g of glucosamine per kg of dry matter.
